(19) United States
(12) Patent Application Publication
(10) Pub. No.: US 2011/0015387 A1
(43) Pub. Date: Jan. 20, 2011

(54) METHOD FOR THE DEPOLYMERIZATION OF CELLULOSE

(75) Inventors: Ferdi Schuth, Mülheim an der Ruhr (DE); Roberto Rinaldi, Mülheim an der Ruhr (DE); Regina Palkovitz, Mülheim an der Ruhr (DE)

Correspondence Address: Briscoe, Kurt G.;Norris McLaughlin & Marcus, PA, 875 Third Avenue, 8th Floor, New York, NY, 10022, US

(73) Assignee: STUDIENGESELLSCHAFT KOHLE MBH, Mulheim an der Ruhr (DE)

(21) Appl. No.: 12/920,863

(22) PCT Filed: Mar. 16, 2009
(86) PCT No.: PCT/DE09/00339

§ 371 Date: Sep. 3, 2010

(30) Foreign Application Priority Data
Mar. 18, 2008 (DE) 10 2008 014 735.4

(51) Int. Cl. C07H 1/00

(52) U.S. Cl. 536/124

(57) ABSTRACT

A process for the depolymerization of cellulose, in which a solution of cellulose in an ionic liquid is brought into contact with a solid acid as catalyst, is claimed. The cellulose can be depolymerized within a short reaction time to form a low molecular weight or oligomeric reaction mixture having a narrow molecular weight distribution (low polydispersity, d, defined as ratio of P_{w} to Pₙ).

The present invention relates to a process for the depolymerization of cellulose, in which the cellulose is reacted in an ionic liquid in the presence of catalysts.

Cellulose is the main constituent of the cell walls of plants and, with an occurrence of about 1200 billion metric tons, is the most abundant organic polymer compound on earth and is a substantial constituent of the biomass. It is therefore also the most abundant polysaccharide. Chemically, cellulose is an unbranched polysaccharide which consists of from several hundred to ten thousand β-D-glucose molecules. The number of β-D-glucose units is defined as the degree of polymerization of the cellulose (P_{w}—weight average of the degree of polymerization, Pₙ—number average of the degree of polymerization). It is an important industrial raw material which is used as basic material in the paper industry or in the clothing industry as viscose, cotton fibers or linen. A further important field of application is the building industry where cellulose derivatives such as methylcellulose are used as flow improvers, etc. Further fields of application are the production of cellophane or the development of renewable automobile fuels, e.g. cellulose ethanol which is produced from vegetable biomass. Furthermore, cellulose derivatives are used as additives in the food and pharmaceutical industries.

Cellulose is insoluble in water and in most organic solvents. It has a certain solubility in toxic solvents such as CS₂, amines, morpholines, concentrated mineral acids, molten salts and in cuprammonium solutions. Solvents used commercially at present are, for example, N-methylmorpholine N-oxide and CS₂.

It is also possible to dissolve cellulose purely physically in an ionic liquid. Chemical syntheses which are not possible in other solvents can be carried out using the cellulose which has been dissolved in this way.

Some industrial states are pursuing the aim of increasing the proportion of renewable raw materials in the production of typical industrial products such as paints, varnishes, plastics, fibers or medicaments from biomass. For this purpose, it is necessary to digest the biomass, i.e. separate it into its individual constituents, to such a degree that this can then be processed further to give desired products. Without chemical digestion, e.g. hydrolysis of cellulose, the cellulose is not very suitable for enzymatic processes.

Even though paper, textile fibers, packaging materials and inhibitors are already produced from cellulose, it is desirable to make cellulose available as renewable raw material for other applications, too. A prerequisite for this is simplified processability of cellulose.

It is accordingly an object of the present invention to provide a process for the processing of cellulose, in which the cellulose is split into smaller molecular units which can be passed to further processing in a manner known per se.

The present invention accordingly provides a process for the depolymerization of cellulose, in which a solution of cellulose in an ionic liquid is brought into contact with a solid acid as catalyst.

It has surprisingly been found that cellulose can be depolymerized within a short reaction time in an ionic liquid in the presence of a catalyst. This gives a low molecular weight or oligomeric reaction mixture having a narrow molecular weight distribution (low polydispersity, d, defined as the ratio of P_{w} to Pₙ). The pretreatment of cellulose with a heterogeneous acid catalyst in an ionic liquid enables a low molecular weight or oligomeric reaction mixture having a narrow molecular weight distribution to be obtained within a short time. The degree of polymerization of the depolymerized cellulose is usually in the range from 1000 to 30 glucose units. It is in principle also possible to carry out the depolymerization through to the monomeric units. However, the reaction can be stopped earlier, for example when cellulose oligomers are to be processed further and degradation through to the monomers would not be economically feasible.

For the purposes of the present patent application, ionic liquids are organic salts whose melting point is below 180° C., i.e. are liquid at temperatures below 180° C. The melting point is preferably in the range from −50° C. to 150° C., particularly preferably in the range from −20° C. to 120° C. and in particular below 100° C. Examples of cations used are alkylated imidazolium, pyridinium, ammonium or phosphonium ions. As anions, it is possible to employ various ions from simple halide through more complex inorganic ions such as tetrafluoroborates to large organic ions such as trifluororomethanesulfonamide. Examples of suitable ionic Liquids are described in the patent documents US-A1 943,176, WO 03/029329, WO 07/057235.

Cations and anions are present in the ionic liquid. Within the ionic liquid, a proton or an alkyl radical can be transferred from the cation to the anion. An equilibrium of anions, cations and neutral substances formed therefrom can thus be present in the ionic liquid used according to the invention.

Ionic liquids which have alkylated imidazolium, pyridinium, ammonium or phosphonium radicals as cations and halides, inorganic, complex anions such as tetrafluoroborates or thiocyanates and organic anions such as trifluororomethanesulfonamides or carboxylate anions as anions have been found to be particularly useful.

Ionic liquids which are suitable for the process of the invention preferably have

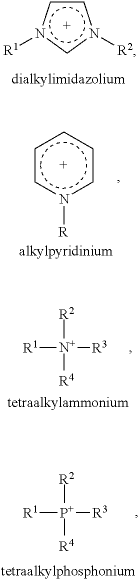

as cations. The anions are preferably selected from among chloride, bromide, nitrate, sulfate, phosphate, tetrafluoroborate, tetrachloroaluminate; tetrachloroferrate (III), hexafluorophosphate, hexafluoroantimonate, carboxylate anions, trifluoromethanesulfonate, alkylphosphate, alkylsulfate, alkylsulfonate, benzenesulfonate, bis(trifluoro-methylsulfonyl)imide, trifluororomethanesulfonamide, thiocyanates. The cations and anions can be combined in any way.

According to the invention, solid acids which represent heterogeneous acid catalysts are used as catalysts. These have the advantage that they are active in solid form and can be separated from the reaction products after the reaction is complete. The solid acids preferably have groups selected from among —SO₃H, —OSO₃H, —PO₂H, —PO(OH)₂ and/or —PO(OH)₃.

In a preferred embodiment, acidic ion exchangers or acidic inorganic metal oxides are used as catalysts. Acidic ion exchanges are, for example, macroporous or mesoporous crosslinked polymers which have acid groups such as —SO₃H on their surface. Further suitable catalysts are, for example, silicon oxide, aluminum oxide, aluminosilicates and zirconium oxide whose surface can be modified further by functionalization with —SO₃H or —OSO₃H groups.

Particularly suitable catalysts are ion exchange resins. The ion exchange resins usually have a surface area of from 1 to 500 m²g⁻¹, in particular from 1 to 150 m²g⁻¹ and preferably from 1 to 41 m²g⁻¹. These ion exchange resins preferably have a pore volume of from 0.002 to 2 cm³g⁻¹, in particular from 0.002 to 0.220 cm³g⁻¹. The average pore diameter is generally from 1 to 100 nm, in particular from 15 to 80 nm and preferably from 24 to 30 nm. Ion exchangers having an ion exchange capacity of from 1 to 10 mmol g⁻¹, in particular from 2.5 to 5.4 mmol g⁻¹, are well suited in the process of the invention.

Examples of suitable commercially available acid catalysts are Nafion® (sulfonated polytetrafluoroethylene (PTFE), DuPont) or Amberlyst® 15 DRY (Rohm and Haas). It is also possible to use mixtures of acid group-containing polymers and inorganic components as catalysts, e.g. mixtures of sulfonated polymers such as sulfonated polytetrafluoroethylene together with nanosize SiO₂, namely a composite.

The reaction can, compared to the prior art, be carried out at relatively low temperatures. The depolymerization occurs in a relatively short reaction time in a temperature range from 50 to 130° C., preferably from 80 to 130° C. The reaction times can be from 0.25 to 5 hours. Longer reaction times are less preferred for economic reasons.

The oligomers obtained from the process of the invention can be separated off from the ionic liquid in a simple manner, for example by filtration. In one possible embodiment, the degradation products of cellulose which are obtained can be precipitated from the ionic liquid by addition of water. Thus, in order to be able to remove the ionic liquid as completely possible, the oligomers may be washed with water, liquid ammonia, dichloromethane, methanol, ethanol or acetone.

The invention is illustrated by the following examples:

### EXAMPLES

### Example 1

5 g of α-cellulose were dissolved in 100 g of 1-butyl-3-methylimidazolium chloride at 100° C. After dissolution of the cellulose, 2 ml of distilled water were added. The solution was stirred at 100° C. for a further 5 hours. In this experiment, no catalyst at all was used. Samples were taken from the reaction mixture every hour during the first 5 hours. 25 ml of water were added to each of the samples, resulting in precipitation of long-chain cellulose units. The precipitated material was separated from the solution by centrifugation and dried overnight at 90° C. The amount of recovered cellulose was determined by weighing of the cellulose samples. These samples were derivatized by means of phenyl isocyanate for GPC analysis.

Table 1 shows the degree of polymerization and the polydispersity of the cellulose obtained as a function of the time of the experiment.

TABLE 1
Depolymerization experiment without addition of catalyst

| Time of | | | | Cellulose |
|---|---|---|---|---|
| experiment (h) | Pₙ | P_{w} | d | recovered (%) |
| 0 | 242 | 1210 | 5.0 | 93 |
| 1.0 | 247 | 1014 | 4.1 | 92 |
| 2.0 | 220 | 1012 | 4.6 | 90 |
| 3.0 | 214 | 1095 | 5.1 | 86 |
| 4.0 | 227 | 948 | 4.2 | 83 |
| 5.0 | 235 | 887 | 3.8 | 96 |

P_{w}—weight average of the degree of polymerization;
Pₙ—number average of the degree of polymerization;
d—polydispersity.

In the experiment without addition of catalyst, about 90% of the cellulose used could be recovered at any time. Only a small change in the degree of polymerization is apparent, while the polydispersity remains virtually unchanged. This result indicates a very low degradation of cellulose in ionic liquid without addition of catalysts. In the aqueous samples, no forms of monosaccharides or disaccharides could be detected.

### Example 2

5 g of α-cellulose were dissolved in 100 g of 1-butyl-3-methylimidazolium chloride at 100° C. After dissolution of the cellulose, 2 ml of distilled water were added. The solution was stirred for a further 15 minutes, and 1 g of Amberlyst 15DRY (commercial product from Rohm & Haas, Germany) was subsequently added to the solution. The depolymerization of the cellulose was carried out at 100° C. Samples were taken from the reaction mixture every 15 minutes during the first hour and then every hour. 25 ml of water were added to each of the samples. The precipitated cellulose was separated off by centrifugation and dried overnight at 90° C. The amount of recovered cellulose was determined by weighing of the cellulose samples. These samples were derivatized by means of phenyl isocyanate for GPC analysis.

Table 2 shows the degree of polymerization and the polydispersity of the cellulose obtained as a function of the reaction time.

TABLE 2
Depolymerization of α-cellulose using Amberlyst 15DRY

| Reaction time | | | | Cellulose |
|---|---|---|---|---|
| (h) | Pₙ | P_{w} | d | recovered (%) |
| 0 | 210 | 830 | 4.0 | 87 |
| 0.25 | 94 | 422 | 4.5 | 88 |
| 0.50 | 64 | 219 | 3.4 | 84 |
| 0.75 | 47 | 127 | 2.7 | 53 |
| 1.0 | 34 | 81 | 2.4 | 65 |
| 1.5 | 23 | 50 | 2.2 | 65 |
| 2.0 | 17 | 33 | 1.9 | 66 |
| 3.0 | 12 | 20 | 1.6 | 58 |
| 4.0 | 10 | 15 | 1.4 | 11 |
| 5.0 | 10 | 12 | 1.3 | 8 |

P_{w}—weight average of the degree of polymerization;
Pₙ—number average of the degree of polymerization;
d—polydisperisity

The results show that α-cellulose dissolved in ionic liquids depolymerizes in the presence of a solid, acid catalyst. The number average degree of polymerization Pₙ and the weight average degree of polymerization P_{w} decrease significantly after a reaction time of one hour, with oligomers (P_{w}=81) having a low polydispersity (d=2.4) being obtained. These oligomers can be separated virtually completely from the ionic liquid by precipitating them by addition of water. The product obtained can, for example, be degraded to form products having an even lower degree of polymerization by means of enzymatic catalysis.

The aqueous reaction solutions were analyzed by means of HPLC to determine their content of sugar molecules (cellobiose, glucose, xylose, arabinose) and subsequent products of sugar degradation (5-hydroxymethylfurfural, levulinic acid, furoic acid, furfuraldehyde). In addition, the total amount of reducing sugars present (TRS—total reducing sugars) was detected in the DNS assay. The results are summarized in Table 3.

TABLE 3
Yield of sugar molecules and subsequent products
of sugar degradation in the reaction solutions

| Reac- | | | | | | | 5- | | |
|---|---|---|---|---|---|---|---|---|---|
| tion | Cbe | Glu | Xyl | Ara | LVA | FA | HMF | FAL | TRS |
| time (h) | (%) | (%) | (%) | (%) | (%) | (%) | (%) | (%) | (%) |
| 0 | 0.00 | 0.00 | 0.00 | 0.00 | 0.00 | 0.00 | 0.01 | 0.00 | 0 |
| 0.25 | 0.00 | 0.00 | 0.00 | 0.00 | 1.41 | 0.00 | 0.05 | 0.02 | 0 |
| 0.50 | 0.00 | 0.00 | 0.00 | 0.00 | 1.45 | 0.00 | 0.07 | 0.03 | 0 |
| 0.75 | 0.00 | 0.00 | 0.91 | 0.89 | 1.26 | 0.00 | 0.08 | 0.04 | 3 |
| 1.0 | 0.00 | 0.00 | 0.90 | 0.96 | 1.65 | 0.00 | 0.08 | 0.05 | 4 |
| 1.5 | 0.39 | 0.85 | 1.06 | 0.97 | 3.39 | 0.00 | 0.10 | 0.06 | 6 |
| 2.0 | 0.39 | 0.84 | 1.07 | 0.96 | 5.18 | 0.00 | 0.12 | 0.09 | 10 |
| 3.0 | 0.47 | 1.01 | 1.25 | 0.95 | 14.02 | 0.00 | 0.20 | 0.17 | 17 |
| 4.0 | 0.57 | 1.29 | 1.58 | 1.02 | 19.47 | 0.01 | 0.36 | 0.33 | 26 |
| 5.0 | 0.73 | 1.70 | 1.87 | 0.96 | 20.87 | 0.01 | 0.60 | 0.51 | 35 |

Cbe—cellobiose;
Glu—glucose;
Xyl—xylose;
Ara—arabinose;
5-HMF—5-hydroxymethylfurfural;
LVA—levulinic acid;
FA—furoic acid;
FAL—furfuraldehyde.

Only a low yield of sugars and subsequent products is observed in the first hour. This indicates selective degradation of the cellulose to form relatively small oligomers. Only after formation of these relatively small oligomers does the degradation proceed to sugars and subsequent products of sugars. The main subsequent product of sugar degradation is levulinic acid. The total amount of furan components makes up less than 1.1% of the total concentration.

### Example 3

5 g of microcrystalline cellulose (cotton linters) were dissolved in 100 g of 1-butyl-3-methylimidazolium chloride at 100° C. After dissolution of the cellulose, 2 ml of distilled water were added. The solution was stirred for a further 15 minutes, and 1 g of Amberlyst 15DRY (commercial product from Rohm & Haas, Germany) was subsequently added to the solution. The depolymerization of the cellulose was carried out at 100° C. Samples were taken from the reaction mixture every 15 minutes during the first hour and then every hour. 25 ml of water were added to each of the samples. The precipitated cellulose was separated off by centrifugation and dried overnight at 90° C. The amount of recovered cellulose was determined by weighing the cellulose samples. These samples were derivatized by means of phenyl isocyanate for the GPC analysis.

Table 4 shows the degree of polymerization and the polydispersity of the cellulose obtained as a function of the reaction time.

TABLE 4
Depolymerization of microcrystalline cellulose using Amberlyst 15DRY.

| Reaction time | | | | Cellulose |
|---|---|---|---|---|
| (h) | Pₙ | P_{w} | d | recovered (%) |
| 0 | 63 | 207 | 3.3 | 90 |
| 0.25 | 61 | 191 | 3.2 | 90 |
| 0.50 | 54 | 161 | 3.0 | 87 |
| 0.75 | 38 | 94 | 2.5 | 90 |
| 1.0 | 32 | 75 | 2.4 | 91 |
| 1.5 | 21 | 44 | 2.1 | 91 |
| 2.0 | 17 | 33 | 1.9 | 81 |
| 3.0 | 13 | 22 | 1.7 | 78 |
| 4.0 | 10 | 14 | 1.4 | 60 |
| 5.0 | 9 | 12 | 1.3 | 48 |

P_{w}—weight average of the degree of polymerization;
Pₙ—number average of the degree of polymerization;
d—polydispersity.

Microcrystalline cellulose is obtained as insoluble residue of the acid-catalyzed hydrolysis of amorphous cellulose constituents and was chosen as substrate because there is at present no process for depolymerizing it. Interestingly, the results show that cellulose dissolved in ionic liquids can be depolymerized in the presence of a solid, acid catalyst. The number average degree of polymerization Pₙ and the weight average degree of polymerization P_{w} decrease significantly after a reaction time of one hour, with oligomers (P_{w}=75) having a low polydispersity (d=2.4) being obtained. These oligomers could be separated virtually completely from the ionic liquid by precipitating them by addition of water. The product obtained can, for example, be degraded to form products having an even lower degree of polymerization by means of enzymatic catalysis.

The aqueous reaction solutions were analyzed for their content of sugar molecules (cellobiose, glucose, xylose, arabinose) and subsequent products of sugar degradation (5-hydroxymethylfurfural, levulinic acid, furoic acid, furfuraldehyde) by HPLC. In addition, the total amount of reducible sugars present (TRS—total reducing sugars) was detected in the DNS assay. The results are summarized in Table 5.

TABLE 5
Yield of sugar molecules and subsequent products
of sugar degradation in the reaction solutions.

| Reac- | | | | | | | 5- | | |
|---|---|---|---|---|---|---|---|---|---|
| tion | Cbe | Glu | Xyl | Ara | LVA | FA | HMF | FAL | TRS |
| time (h) | (%) | (%) | (%) | (%) | (%) | (%) | (%) | (%) | (%) |
| 0 | 0.00 | 0.00 | 0.00 | 0.00 | 4.70 | 0.00 | 0.00 | 0.00 | 0 |
| 0.25 | 0.00 | 0.00 | 0.00 | 0.00 | 7.05 | 0.00 | 0.05 | 0.00 | 0 |
| 0.5 | 0.00 | 0.00 | 0.00 | 0.00 | 6.37 | 0.00 | 0.07 | 0.01 | 0 |
| 0.75 | 0.17 | 0.37 | 0.00 | 0.00 | 4.21 | 0.00 | 0.09 | 0.01 | 3 |
| 1.0 | 0.19 | 0.41 | 0.00 | 0.00 | 4.58 | 0.00 | 0.09 | 0.01 | 2 |
| 1.5 | 0.23 | 0.48 | 0.46 | 0.00 | 6.99 | 0.00 | 0.11 | 0.01 | 4 |
| 2.0 | 0.27 | 0.52 | 0.48 | 0.00 | 9.57 | 0.00 | 0.13 | 0.01 | 5 |
| 3.0 | 0.32 | 0.68 | 0.52 | 0.00 | 12.22 | 0.00 | 0.19 | 0.02 | 10 |
| 4.0 | 0.59 | 1.17 | 0.61 | 0.00 | 27.71 | 0.01 | 0.44 | 0.04 | 21 |
| 5.0 | 0.88 | 1.96 | 0.71 | 0.44 | 26.48 | 0.00 | 0.77 | 0.07 | 27 |

Cbe—cellobiose;
Glu—glucose;
Xyl—xylose;
Ara—arabinose;
5-HMF—5-hydroxymethylfurfural;
LVA—levulinic acid;
FA—furoic acid;
FAL—furfuraldehyde.

Only a low yield of sugars and subsequent products is observed in the first hour. This indicates selective degradation of the cellulose to form relatively small oligomers. Only after formation of these relatively small oligomers does the degradation proceed to sugars and subsequent products of sugars. The main subsequent product of sugar degradation is levulinic acid. The total amount of furan components makes up less than 0.8% of the total concentration.

### Example 4

5 g of SigmaCell cellulose were dissolved in 100 g of 1-butyl-3-methylimidazolium chloride at 100° C. After dissolution of the cellulose, 2 ml of distilled water were added. The solution was stirred for a further 15 minutes, and 1 g of Amberlyst 15DRY (commercial product of Rohm & Haas, Germany) was subsequently added to the solution. The depolymerization of the cellulose was carried out at 100° C. Samples were taken from the reaction mixture every 15 minutes during the first hour and then every hour. 25 ml of water were added to each of the samples. The precipitated cellulose was separated off by centrifugation and dried overnight at 90° C. The amount of recovered cellulose was determined by weighing the cellulose samples. These samples were derivatized by means of phenyl isocyanate for the GPC analysis.

Table 6 shows the degree of polymerization and the polydispersity of the cellulose obtained as a function of the reaction time.

TABLE 6
Depolymerization of SigmaCell cellulose using Amberlyst 15DRY

| Reaction time | | | | Cellulose recovered |
|---|---|---|---|---|
| (h) | Pₙ | P_{w} | d | (%) |
| 0 | 132 | 647 | 4.9 | 90 |
| 0.25 | 104 | 480 | 4.6 | 87 |
| 0.50 | 86 | 358 | 4.1 | 84 |
| 0.75 | 68 | 205 | 3.0 | 75 |
| 1.0 | 54 | 138 | 2.5 | 74 |
| 1.5 | 37 | 84 | 2.3 | 56 |
| 2.0 | 26 | 56 | 2.1 | 43 |
| 3.0 | 17 | 31 | 1.8 | 49 |
| 4.0 | 14 | 21 | 1.6 | 64 |
| 5.0 | 12 | 17 | 1.4 | 50 |

P_{w}—weight average of the degree of polymerization;
Pₙ—number average of the degree of polymerization;
d—polydispersity.

SigmaCell cellulose is obtained as a product of the mechanical digestion of cotton linters. The results show that cellulose dissolved in ionic liquids can be depolymerized in the presence of a solid, acid catalyst. The number average degree of polymerization Pₙ and the weight average degree of polymerization P_{w} decrease significantly after a reaction time of one hour, with oligomers (P_{w}=138) having a low polydispersity (d=2.5) being obtained. These oligomers can be separated virtually completely from the ionic liquid by precipitating them by addition of water. The product obtained can, for example, be degraded to form products having an even lower degree of polymerization by means of enzymatic catalysis.

The aqueous reaction solutions were analyzed by means of HPLC to determine their content of sugar molecules (cellobiose, glucose, xylose, arabinose) and subsequent products of sugar degradation (5-hydroxymethylfurfural, levulinic acid, furoic acid, furfuraldehyde). In addition, the total amount of reducing sugars present (TRS—total reducing sugars) was detected in the DNS assay. The results are summarized in Table 7.

TABLE 7
Yield of sugar molecules and subsequent products
of sugar degradation in the reaction solutions.

| Reac- | | | | | | | 5- | | |
|---|---|---|---|---|---|---|---|---|---|
| tion | Cbe | Glu | Xyl | Ara | LVA | FA | HMF | FAL | TRS |
| time (h) | (%) | (%) | (%) | (%) | (%) | (%) | (%) | (%) | (%) |
| 0 | 0.00 | 0.00 | 0.00 | 0.00 | 0.00 | 0.00 | 0.00 | 0.00 | 0 |
| 0.25 | 0.00 | 0.00 | 0.00 | 0.00 | 2.43 | 0.00 | 0.07 | 0.02 | 0 |
| 0.50 | 0.00 | 0.00 | 0.00 | 0.00 | 3.01 | 0.00 | 0.07 | 0.02 | 0 |
| 0.75 | 0.00 | 0.77 | 0.00 | 0.00 | 2.56 | 0.00 | 0.08 | 0.03 | 3 |
| 1.0 | 0.00 | 0.72 | 0.84 | 0.00 | 2.74 | 0.00 | 0.09 | 0.03 | 4 |
| 1.5 | 0.00 | 0.72 | 0.86 | 0.00 | 3.86 | 0.00 | 0.09 | 0.04 | 3 |
| 2.0 | 0.38 | 0.85 | 0.96 | 0.00 | 6.28 | 0.00 | 0.10 | 0.05 | 4 |
| 3.0 | 0.43 | 0.94 | 1.08 | 0.00 | 10.95 | 0.00 | 0.14 | 0.08 | 8 |
| 4.0 | 0.50 | 1.05 | 1.22 | 0.00 | 17.56 | 0.01 | 0.22 | 0.15 | 14 |
| 5.0 | 0.59 | 1.33 | 1.44 | 0.00 | 20.60 | 0.00 | 0.31 | 0.21 | 20 |

Cbe—cellobiose;
Glu—glucose;
Xyl—xylose;
Ara—arabinose;
5-HMF—5-hydroxymethylfurfural;
LVA—levulinic acid;
FA—furoic acid;
FAL—furfuraldehyde.

Only a low yield of sugars and subsequent products is observed in the first hour. This indicates selective degradation of the cellulose to form relatively small oligomers. Only after formation of these relatively small oligomers does the degradation proceed to sugars and subsequent products of sugars. The main subsequent product of sugar degradation is levulinic acid. The total amount of furan components makes up less than 0.5% of the total concentration.

### Example 5

5 g of α-cellulose were dissolved in 100 g of 1-butyl-3-methylimidazolium chloride at 100° C. After dissolution of the cellulose, 2 ml of distilled water were added. The solution was stirred for a further 15 minutes, and 0.9 g of p-toluenesulfonic acid was subsequently added to the solution. The depolymerization of the cellulose was carried out at 100° C. Samples were taken from the reaction mixture every 15 minutes during the first hour and then every hour. 25 ml of water were added to each of the samples. The precipitated cellulose was separated off by centrifugation and dried overnight at 90° C. The amount of recovered cellulose was determined by weighing the cellulose samples. These samples were derivatized by means of phenyl isocyanate for the GPC analysis.

Table 8 shows the degree of polymerization and the polydispersity of the cellulose obtained as a function of the reaction time.

TABLE 8
Depolymerization of α-cellulose using p-toluenesulfonic acid

| Reaction | | | | Cellulose |
|---|---|---|---|---|
| time (h) | Pₙ | P_{w} | d | (recovered (%)) |
| 0 | 210 | 830 | 4.0 | 81 |
| 0.10 | 65 | 198 | 3.0 | — |
| 0.25 | 45 | 107 | 2.4 | 80 |
| 0.50 | 34 | 73 | 2.2 | 68 |
| 0.75 | 26 | 52 | 2.0 | 67 |
| 1.0 | 22 | 44 | 2.0 | 61 |
| 1.5 | 16 | 29 | 1.8 | 73 |
| 2.0 | 14 | 22 | 1.6 | 67 |
| 3.0 | 11 | 16 | 1.4 | 54 |
| 4.0 | 10 | 13 | 1.3 | 39 |
| 5.0 | 9 | 11 | 1.2 | 18 |

P_{w}—weight average of the degree of polymerization;
Pₙ—number average of the degree of polymerization;
d—polydispersity.

The results show that cellulose dissolved in ionic liquids depolymerizes in the presence of p-tofuenesulfonic acid (homogeneous acid catalyst). The number average degree of polymerization Pₙ and the weight average degree of polymerization P_{w} decrease significantly after a reaction time of one hour, with oligomers (P_{w}=44) having a low polydispersity (d=2.0) being obtained. These oligomers can be separated virtually completely from the ionic liquid by precipitating them by addition of water. However, the product obtained requires a neutralization step. The catalyst can be separated from the reaction mixture only with difficulty.

The aqueous reaction solutions were analyzed by means of HPLC to determine their content of sugar molecules (cellobiose, glucose, xylose, arabinose) and subsequent products of sugar degradation (5-hydroxymethylfurfural, levulinic acid, furoic acid, furfuraldehyde). In addition, the total amount of reducing sugars present (TRS—total reducing sugars) was detected in the DNS assay. The results are summarized in Table 9.

TABLE 9
Yield of sugar molecules and subsequent products
of sugar degradation in the reaction solutions

| Reac- | | | | | | | 5- | | |
|---|---|---|---|---|---|---|---|---|---|
| tion | Cbe | Glu | Xyl | Ara | LVA | FA | HMF | FAL | TRS |
| time (h) | (%) | (%) | (%) | (%) | (%) | (%) | (%) | (%) | (%) |
| 0 | 0.00 | 0.00 | 0.00 | 0.00 | 0.00 | 0.00 | 0.00 | 0.00 | 0 |
| 0.10 | 0.00 | 0.00 | 0.00 | 0.92 | 1.04 | 0.00 | 0.01 | 0.01 | 0 |
| 0.25 | 0.00 | 0.00 | 0.90 | 0.94 | 1.79 | 0.00 | 0.01 | 0.02 | 0 |
| 0.50 | 0.38 | 0.74 | 0.92 | 0.97 | 2.76 | 0.00 | 0.01 | 0.03 | 3 |
| 0.75 | 0.36 | 0.82 | 1.01 | 0.95 | 0.77 | 0.00 | 0.02 | 0.03 | 7 |
| 1.0 | 0.38 | 0.83 | 1.09 | 0.92 | 1.38 | 0.00 | 0.03 | 0.04 | 7 |
| 1.5 | 0.43 | 0.91 | 1.15 | 0.96 | 1.87 | 0.01 | 0.06 | 0.09 | 11 |
| 2.0 | 0.48 | 1.04 | 1.29 | 0.92 | 3.25 | 0.00 | 0.10 | 0.13 | 16 |
| 3.0 | 0.57 | 1.28 | 1.61 | 0.96 | 16.46 | 0.00 | 0.22 | 0.25 | 24 |
| 4.0 | 0.73 | 1.64 | 1.95 | 1.00 | 19.25 | 0.00 | 0.50 | 0.48 | 33 |
| 5.0 | 0.93 | 2.36 | 2.17 | 0.99 | 42.22 | 0.01 | 0.86 | 0.72 | 41 |

Cbe—cellobiose;
Glu—glucose;
Xyl—xylose;
Ara—arabinose;
5-HMF—5-hydroxymethylfurfural;
LVA—levulinic acid;
FA—furoic acid;
FAL—furfuraldehyde.

Sugars can be detected in the reaction solution after only 0.5 h. Their concentration increases continuously during the course of the reaction. In addition, subsequent products of sugar degradation are formed at the same time. The main subsequent product of sugar degradation is levulinic acid. The total amount of furan components makes up 1.6% of the total concentration

### Example 6

5 g of α-cellulose were dissolved in 100 g of 1-butyl-3-methylimidazolium chloride at 100° C. 2 ml of distilled water were added to the solution and the solution was stirred for a further 15 minutes. The solution was divided into samples of 10 g each. 0.1 g of various solid acid catalysts were subsequently added to each of the samples. The samples were reacted at 100° C. for 1 hour. 25 ml of water were added to each of the samples. The precipitated cellulose was separated off by centrifugation and dried overnight at 90° C. The amount of recovered cellulose was determined by weighing the cellulose samples. These samples were derivatized by means of phenyl isocyanate for the GPC analysis.

The degree of polymerization and the polydispersity of the cellulose obtained after a reaction time of one hour are shown in Table 10.

TABLE 10
Catalyst comparison for the depolymerization of α-cellulose.

| | | | | Cellulose |
|---|---|---|---|---|
| Catalyst | Pₙ | P_{w} | d | (recovered (%)) |
| Blank | 211 | 1623 | 7.7 | 86 |
| Amberlyst 15DRY | 34 | 82 | 2.4 | 65 |
| Amberlyst 35 | 35 | 88 | 2.5 | 23 |
| Amberlyst 70 | 209 | 1489 | 7.1 | — |
| Nafion | 230 | 1571 | 6.8 | — |
| Aluminum oxide | 193 | 1171 | 6.1 | 84 |
| Sulfated zirconia | 250 | 1482 | 5.9 | — |
| Silica-alumina | 190 | 1920 | 10.1 | 100 |
| Zeolite Y | 210 | 1989 | 9.4 | 100 |
| ZSM-5 | 166 | 2055 | 12.3 | 100 |

P_{w}—weight average of the degree of polymerization;
Pₙ—number average of the degree of polymerization;
d—polydispersity.

The aim of this study was to screen the potential of various heterogeneous acid catalysts for cellulose degradation. The potential of the catalysts was evaluated by means of the course of the number average degree of polymerization Pₙ and the weight average degree of polymerization P_{w}. Amberlyst 35 shows a potential comparable to that of Amberlyst 15DRY in the depolymerization of cellulose. On the other hand, Amberlyst 70 and Nafion led to only small changes in the degree of polymerization of the cellulose. The inorganic metal oxides aluminum oxide and sulfated zirconium dioxide resulted in an average degradation of the cellulose, while aluminosilicates, e.g. silica-alumina, zeolite Y and ZSM-5, even increase the apparent degree of polymerization P_{w}.

### Example 7

5 g of wood were dissolved in 100 g of 1-butyl-3-methylimidazolium chloride at 100° C. After dissolution of the cellulose, 2 ml of distilled water were added. The solution was stirred for a further 15 minutes, and 1 g of Amberlyst 15DRY (commercial product of Rohm & Haas, Germany) was subsequently added to the solution. The depolymerization of the cellulose was carried out at 100° C. Samples were taken from the reaction mixture every 15 minutes during the first hour and then every hour. 25 ml of water were added to each of the samples. The precipitated cellulose was separated off by centrifugation. Table 2 shows the degree of polymerization of the cellulose obtained as a function of the reaction time. These samples were derivatized by means of phenyl isocyanate for the GPC analysis.

TABLE 11
Depolymerization of wood using Amberlyst 15DRY.

| Reaction time (h) | Pₙ | P_{w} |
|---|---|---|
| 0 | 1928 | 611 |
| 0.5 | 577 | 284 |
| 1.0 | 288 | 69 |
| 2.0 | 153 | 59 |
| 3.0 | 44 | 21 |

P_{w}—weight average of the degree of polymerization;
Pₙ—number average of the degree of polymerization.

### Example 8

5 g of α-cellulose were dissolved in 100 g of 1-butyl-3-methylimidazolium chloride at 100° C. After dissolution of the cellulose, 2 ml of distilled water were added. The solution was stirred for a further 15 minutes, and 1 g of Amberlyst 15DRY (commercial product of Rohm & Haas, Germany) was subsequently added to the solution. The depolymerization of the cellulose was carried out at 80° C. Samples were taken from the reaction mixture every 15 minutes during the first hour and then every hour. 25 ml of water were added to each of the samples. The precipitated cellulose was separated off by centrifugation and dried overnight at 90° C. These samples were derivatized by means of phenyl isocyanate for the GPC analysis.

Table 12 shows the degree of polymerization of the cellulose obtained as a function of the reaction time.

TABLE 12
Depolymerization of α-cellulose using Amberlyst 15DRY at 80° C.

| Reaction time (h) | Pₙ | P_{w} |
|---|---|---|
| 0 | 1173 | 242 |
| 0.25 | 990 | 226 |
| 0.50 | 857 | 210 |
| 0.75 | 898 | 210 |
| 1.0 | 855 | 193 |
| 1.5 | 800 | 195 |
| 2.0 | 536 | 144 |
| 3.0 | 323 | 110 |
| 4.0 | 226 | 89 |
| 5.0 | 161 | 69 |

P_{w}—weight average of the degree of polymerization;
Pₙ—number average of the degree of polymerization.

### Example 9

5 g of α-cellulose were dissolved in 100 g of 1-butyl-3-methylimidazolium chloride at 100° C. After dissolution of the cellulose, 2 ml of distilled water were added. The solution was stirred for a further 15 minutes, and 1 g of Amberlyst 15DRY (commercial product of Rohm & Haas, Germany) was subsequently added to the solution. The depolymerization of the cellulose was carried out at 120° C. Samples were taken from the reaction mixture every 15 minutes during the first hour and then every hour. 25 ml of water were added to each of the samples. The precipitated cellulose was separated off by centrifugation and dried overnight at 90° C. These samples were derivatized by means of phenyl isocyanate for the GPC analysis.

Table 13 shows the degree of polymerization of the cellulose obtained as a function of the reaction time.

TABLE 13
Depolymerization of α-cellulose using Amberlyst 15DRY at 120° C.

| Reaction time (h) | Pₙ | P_{w} |
|---|---|---|
| 0 | 1082 | 210 |
| 0.25 | 689 | 155 |
| 0.50 | 109 | 47 |
| 0.75 | 46 | 25 |
| 1.0 | 25 | 16 |
| 1.5 | 16 | 12 |

P_{w}—weight average of the degree of polymerization;
Pₙ—number average of the degree of polymerization.

### Example 10

5 g of α-cellulose were dissolved in 100 g of 1-butyl-3-methylimidazolium chloride at 100° C. After dissolution of the cellulose, 2 ml of distilled water were added. The solution was stirred for a further 15 minutes, and 0.5 g of Amberlyst 15DRY (commercial product of Rohm & Haas, Germany) was subsequently added to the solution. The depolymerization of the cellulose was carried out at 100° C. Samples were taken from the reaction mixture every 15 minutes during the first hour and then every hour. 25 ml of water were added to each of the samples. The precipitated cellulose was separated off by centrifugation and dried overnight at 90° C. These samples were derivatized by means of phenyl isocyanate for the GPC analysis.

Table 14 shows the degree of polymerization of the cellulose obtained as a function of the reaction time.

TABLE 14
Depolymerization of α-cellulose using Amberlyst 15DRY

| Reaction time (h) | Pₙ | P_{w} |
|---|---|---|
| 0 | 880 | 186 |
| 0.25 | 797 | 181 |
| 0.50 | 770 | 181 |
| 0.75 | 710 | 165 |
| 1.0 | 673 | 160 |
| 1.5 | 614 | 149 |
| 2.0 | 555 | 144 |
| 3.0 | 462 | 130 |
| 4.0 | 353 | 113 |
| 5.0 | 288 | 96 |

P_{w}—weight average of the degree of polymerization;
Pₙ—number average of the degree of polymerization.

### Example 11

5 g of α-cellulose were dissolved in 100 g of 1-butyl-3-methylimidazolium chloride at 100° C. After dissolution of the cellulose, 2 ml of distilled water were added. The solution was stirred for a further 15 minutes, and 2 g of Amberlyst 15DRY (commercial product of Rohm & Haas, Germany) were subsequently added to the solution. The depolymerization of the cellulose was carried out at 100° C. Samples were taken from the reaction mixture every 15 minutes during the first hour and then every hour. 25 ml of water were added to each of the samples. The precipitated cellulose was separated off by centrifugation and dried overnight at 90° C. These samples were derivatized by means of phenyl isocyanate for the GPC analysis.

Table 15 shows the degree of polymerization of the cellulose obtained as a function of the reaction time.

TABLE 15
Depolymerization of α-cellulose using Amberlyst 15DRY

| Reaction time (h) | Pₙ | P_{w} |
|---|---|---|
| 0 | 1147 | 235 |
| 0.25 | 91 | 44 |
| 0.50 | 47 | 24 |
| 0.75 | 34 | 20 |
| 1.0 | 22 | 15 |
| 1.5 | 15 | 11 |
| 2.0 | 13 | 10 |

P_{w}—weight average of the degree of polymerization;
Pₙ—number average of the degree of polymerization.

### Example 12

5 g of α-cellulose were dissolved in 100 g of 1-butyl-3-methylimidazolium chloride at 100° C. After dissolution of the cellulose, 2 ml of distilled water were added. The solution was stirred for a further 15 minutes, and 1 g of Amberlyst 15DRY (commercial product of Rohm & Haas, Germany) was subsequently added to the solution. The depolymerization of the cellulose was carried out at 100° C. Samples were taken from the reaction mixture every 15 minutes during the first hour and then every hour.

The depolymerization product obtained was precipitated by addition of liquid ammonia.

### Example 13

5 g of α-cellulose were dissolved in 100 g of 1-butyl-3-methylimidazolium chloride at 100° C. After dissolution of the cellulose, 2 ml of distilled water were added. The solution was stirred for a further 15 minutes, and 1 g of Amberlyst 15DRY (commercial product of Rohm & Haas, Germany) was subsequently added to the solution. The depolymerization of the cellulose was carried out at 100° C. Samples were taken from the reaction mixture every 15 minutes during the first hour and then every hour.

The depolymerization product obtained was precipitated by addition of dichloromethane.

### Example 14

5 g of α-cellulose were dissolved in 100 g of 1-butyl-3-methylimidazolium chloride at 100° C. After dissolution of the cellulose, 2 ml of distilled water were added. The solution was stirred for a further 15 minutes, and 1 g of Amberlyst 15DRY (commercial product of Rohm & Haas, Germany) was subsequently added to the solution. The depolymerization of the cellulose was carried out at 100° C. Samples were taken from the reaction mixture every 15 minutes during the first hour and then every hour.

The depolymerization product obtained was precipitated by addition of methanol.

### Example 15

5 g of α-cellulose were dissolved in 100 g of 1-butyl-3-methylimidazolium chloride at 100° C. After dissolution of the cellulose, 2 ml of distilled water were added. The solution was stirred for a further 15 minutes, and 1 g of Amberlyst 15DRY (commercial product of Rohm & Haas, Germany) was subsequently added to the solution. The depolymerization of the cellulose was carried out at 100° C. Samples were taken from the reaction mixture every 15 minutes during the first hour and then every hour.

The depolymerization product obtained was precipitated by addition of ethanol.

1. A process for the depolymerization of cellulose, said process comprising bringing a solution of cellulose in an ionic liquid into contact with a solid acid as catalyst.

2. The process as claimed in claim 1, wherein the catalyst has one or more acid groups selected from the group consisting of —SO₃H, —OSO₃H, —PO₂H, —PO(OH)₂ and —PO(OH)₃.

3. The process as claimed in claim 1, wherein the acid is selected from the group consisting of ion exchangers and acidic inorganic metal oxides.

4. The process according to claim 3, wherein the ion exchanger is an ion exchanger resin.

5. The process as claimed in claim 4, wherein the ion exchange resin has a surface area of from 1 to 500 m²g⁻¹.

6. The process as claimed in claim 4, wherein the ion exchange resin has a pore volume of from 0.002 to 2 cm³g⁻¹.

7. The process as claimed in claim 4, wherein the ion exchange resin has an average pore diameter of from 1 to 100 nm.

8. The process as claimed in claim 4, wherein the ion exchange capacity is from 1 to 10 mmol g⁻¹.

9. The process as claimed in claim 1, wherein the ionic liquid has a melting point below 180° C.

10. The process as claimed in claim 1, wherein the cations in the ionic liquid are selected from the group consisting of alkylated imidazolium, pyridinium, ammonium and phosphonium cations.

11. The process as claimed in claim 10, wherein the cation in the ionic liquid is selected from the group consisting of

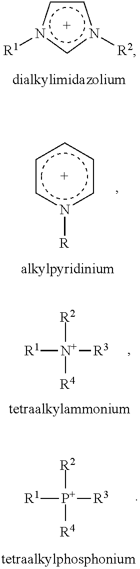

12. The process as claimed in claim 1, wherein the anions in the ionic liquid are selected from the group consisting of inorganic anions and organic ions.

13. The process as claimed in claim 1, wherein the process is carried out at a temperature in the range from 50° C. to 130° C.
